(19) United States
(12) Patent Application Publication
(10) Pub. No.: US 2017/0196927 A1
(43) Pub. Date: Jul. 13, 2017

(54) COMPOSITION FOR ARTHRITIS, MOBILITY AND DELAY AGEING

(71) Applicant: Mars, Incorporated, McLean, VA (US)

(72) Inventor: Samuel SERISIER, AIRMAGUES (FR)

(73) Assignee: Mars, Incorporated, McLean, VA (US)

(21) Appl. No.: 15/313,516

(22) PCT Filed: May 21, 2015
(86) PCT No.: PCT/EP2015/061327

§ 371 Date: Nov. 22, 2016

(30) Foreign Application Priority Data
May 23, 2014 (EP) 14305772.7
Aug. 21, 2014 (GB) 1414910.8

(51) Int. Cl. A61K 36/82; A61K 31/737; A23K 20/147; A23K 50/40; A23K 20/10; A61K 38/39; A61K 9/00

(52) U.S. Cl. CPC A61K 36/82; A61K 38/39; A61K 31/737; A61K 9/0056; A23K 50/40; A23K 20/10; A23K 20/147

(57) ABSTRACT

The present invention relates to a composition comprising green tea extract, collagen and chondroitin. Such a composition can be used for use in treating or preventing arthritis, for increasing life expectancy in an animal, for preventing signs of aging, for use in preserving mobility or preventing decline in mobility in an animal and in a method for making such compositions. The composition can be also used in a method of treating arthritis, a method of increasing life expectancy, a method of preserving mobility or preventing decline in mobility or a method of delaying aging.

The present invention relates to a composition comprising green tea extract, collagen and chondroitin. Such a composition can be used for use in treating or preventing arthritis, for increasing life expectancy in an animal, for use in preserving mobility or preventing decline in mobility in an animal and in a method for making such compositions. The composition can be also used in a method of treating arthritis, a method of increasing life expectancy, a method of preserving mobility or preventing decline in mobility or a method of delaying aging.

The maintenance and improvement of animal health is a constantly ongoing aim in the art. Health includes that of any animal, mammal, in particular a cat, dog or a human.

Today's veterinary medical advances provide pet owners with the ability to extend a pet's life span and improve quality of life, whether the pet falls ill or not. Pet owners will go to great lengths to extend the life of a cherished pet. Pet owners often have a high degree of attachment or extreme commitment to a pet or rely on the animal for primary emotional support. Therefore there is a need for products to extend the life span and ensure a high quality of life for pets and indeed other animals, including humans.

Arthritis is a common problem in pet animals and humans. Treatment of osteoarthritis in dogs can be either medical or surgical which in serious cases can include hip replacements. Generally osteoarthritis in dogs will worsen with time but certain medications can slow down the process. Treatment can include administration of anti-inflammatory drugs. The same applies to humans. Unfortunately these drugs have undesirable side effects and surgery is lengthy and costly.

A food that could prolong an animal's life is highly desirable. In addition, there is a continued need for new methods and compositions that can be used to treat osteoarthritis in animals and in particular for food compositions effective in managing these conditions.

The present invention relates to compositions and their uses.

According to a first aspect of the invention, there is provided a composition comprising green tea extract, collagen and chondroitin.

Green tea extract refers to a herbal derivative from green tea leaves (Camellia sinensis). Green tea extracts can be created by soft infusions, soft extracts, dry extracts, and partly purified extracts techniques. Green tea extract can comprise green tea catechins (GTC), epigallocatechin (EGC), epicatechin gallate (ECG), epigallocatechin gallate (EGCG) and flavonoids such as kaempferol, quercetin and myricetin.

Collagen includes hydrolysed collagen, fibrillary collagen and Collagen Type I to XVIII. Hydrolysed collagen is particularly preferred, especially in combination with the green tea extract and the chondroitin sulphate.

Chondroitin is a chondrin derivative. Chondroitin includes chondroitin sulphate.

Any one or more of the ingredients may be present in the following ranges: green tea extract at 15 mg to 5 g, hydrolysed collagen at 50 mg to 17 g, and chondroitin at 10 mg to 4 g. The ingredients may be present in the following ranges: green tea extract at 15 mg to 1 g, hydrolysed collagen at 50 mg to 1 g, and chondroitin at 10 mg to 1 g. The ingredients may be present in the following ranges: green tea extract at 15 mg to 500 mg, hydrolysed collagen at 50 mg to 500 mg, and chondroitin at 10 mg to 500 mg. The ingredients may be present in the following ranges: green tea extract at 1 g to 5 g, hydrolysed collagen at 5 g to 17 g, and chondroitin at 1 g to 4 g. The ingredients may be present in the following ranges: green tea extract at 1 g to 3 g, hydrolysed collagen at 8 g to 14 g, and chondroitin at 1 g to 3 g. The ingredients may be present in the following ranges: green tea extract at 200 mg to 2 g, hydrolysed collagen at 500 mg to 5 g, and chondroitin at 200 mg to 2 g.

These amounts of the ingredients green tea extract, collagen and chondroitin can be provided as a total per day. The amount will take into account the size of the animal. A desired amount of 30 mg/kg/day of green tea extract will be in the range of around 15 mg for a small dog (around 0.5 kg) to around 5 g for a large dog (166 kg). For collagen a desired amount may be 100 mg/kg/day and will be in the range of around 50 mg for a small dog (around 0.5 kg) to around 17 g for a large dog (166 kg). For chondroitin, a desired amount may be 20 mg/kg/day and will be in the range of around 10 mg for a small dog (around 0.5 kg) to around 3 g for a large dog (166 kg)

Dog weight can range from 1 kg to120 kg, from 5 kg to 100 kg, from 10 kg to 90 kg, from 15 kg to 60 kg, from 20 kg to 40 kg, from 25 kg to 35 kg.

Suitable ranges also include from 20 mg/kg/day green tea extract to 40 mg/kg/day. Around 0.2, 0.25, 0.3, 0.35% of a diet is suitable (especially for dogs).

Suitable ranges also include from 50 mg/kg/day to 150 mg/kg/day collagen. Around 0.5, 0.8, 0.9, 1.0% of a diet is suitable (especially for dogs).

Suitable ranges also include 150 mg/kg/day to 250 mg/kg/day chondroitin. Around 0.1, 0.15, 0.16, 0.7, 0.2% of a diet is suitable (especially for dogs).

A property of the composition as defined in the first aspect of the invention is that it is thermally stable enough to be used and therefore desirable when manufacturing food.

The composition may be in the form of a food. The food may be a dry, semi-moist or a moist product. Wet food includes food which is sold in tins, or pouches and has a moisture content of 70 to 90%. Dry food includes food having a similar composition, but with 5 to 15% moisture and presented as small biscuit—like kibbles. Semi moist products have a moisture range of from 16% to 69%. The amount of moisture in any product may influence the type of packaging which can be used or is required.

The food may be manufactured by mixing together ingredients and pulverising in order to make consistent dough that can be cooked. The process of creating a dry pet food is usually done by baking and/or extruding. The dough is typically fed into a machine called an expander, which uses pressurized steam or hot water to cook the ingredients. While inside the expander, the dough is under extreme pressure and high temperatures. The dough is then pushed through a die (specifically sized hole) and then cut off using a knife. The puffed dough pieces are made into kibble by passing it through a dryer so that any remaining moisture is drawn out. The kibble can then be sprayed with fats, oils, minerals and vitamins and optionally sealed into packages.

To manufacture canned food, meat products are first rendered, or processed, to separate the water, fat and protein components. The meat is then ground and cooked and then mixed with other ingredients. The finished product is filled into cans, for a shelf life of three to five years. The cans are vacuum packed.

The composition may be in the form of a food supplement. The composition may be presented as a powder or crumbs, including a white powder or solid form. A powder is useful to sprinkle on the main food of the animal. Other forms include solid pellets, granules, tablets or a liquid.

The food is preferably packaged. In this way, the consumer is able to identify, from the packaging, the ingredients in the food product and confirm that it is suitable for the particular pet in question. The packaging may be metal (usually in the form of a tin or flexifoil), plastic, paper or card.

The composition as in the form of a pet food product can encompasses any product which a pet consumes in its diet. Thus, the invention covers standard food products as well as pet food snacks (for example, snack bars, biscuits and sweet products). The food product is preferably a cooked product. It may incorporate meat or animal derived material (such as beef, chicken, turkey, lamb, fish, blood plasma, marrow bone etc or one or more thereof). The product alternatively may be meat free (preferably including a meat substitute such as soya, maize gluten or a soya product) in order to provide a protein source. The product may contain additional protein sources such as soya protein concentrate, milk proteins, gluten etc. The product may also contain a starch source such as one or more grains (e.g. wheat, corn, rice, oats, barley etc), or may be starch free. The product may include fibre such as chicory, sugar beet pulp, etc and/or components such as inulin, fructooligosaccharides, probiotics, most preferably, the combined ingredients of the pet food product according to the invention provide all the recommended vitamins and minerals for the particular animal in question (a complete and balanced food) for example as described in National Research Council, 1985, Nutritional Requirements for dogs, National Academy Press, Washington D.C. or Association of America Feed Control Officials, Official Publication 1996.

The food product can be made according to any method known in the art, such as in Waltham Book of Dog and Cat Nutrition, Ed. ATB Edney, Chapter by A. Rainbird, entitled “A Balanced Diet” in pages 57 to 74 Pergamon Press Oxford.

The composition of the first aspect of the invention is particularly for use in preventing or treating arthritis in an animal, in particular in a cat or a dog. Arthritis includes osteoarthritis, rheumatoid arthritis, psoriatic arthritis, septic arthritis, ankylosing spondylitis (AS), and systemic lupus erythematosus.

The composition of the first aspect of the invention is particularly for use in increasing life expectancy in an animal, in particular a cat or a dog. Increased life expectancy can be measured as extending the life span of the animal. Other effects of the composition include preserving vitality, health, physical vigour, quality of life, and delaying the signs of aging. Physical vigour includes the pet having energy and being active. Physical vigour can be measured by the animals overall energy level, mobility, appetite and playfulness.

The composition of the first aspect of the invention is particularly for use in preserving mobility or preventing decline in mobility in an animal, in particular in a cat or a dog. Often mobility in animals, such as pets decreases because of joint problems. One of the causes of joint problems is the cartilage wears away faster than it can be replaced by the body. When the cartilage wears away the joints become swollen and painful thus creating difficulties with mobility. Signs of decreased mobility in a pet include lifestyle and behavioural changes such as a reduced ability or willingness to jump or run/walk, increased sleep, and difficulty going up and down stairs.

The composition of the first aspect of the invention is particularly for use in delaying aging in an animal, in particular in a cat or a dog. Signs of aging can include a general decrease in energy, slower movements, reduced hearing, reduced eyesight, etc.

According to a second aspect of the invention, there is provided a method of treating arthritis in an animal, in particular a cat or a dog comprising administering, to said animal, the composition as defined in the first aspect of the invention. Arthritis includes osteoarthritis, rheumatoid arthritis, psoriatic arthritis, septic arthritis, ankylosing spondylitis (AS), and systemic lupus erythematosus. The method may be prophylactic or therapeutic.

According to a third aspect of the invention, there is provided a method of increasing life expectancy in an animal, in particular in a cat or a dog comprising administering, to said animal, the composition as defined in the first aspect of the invention. Increased life expectancy can be measured as extending the life span of the animal. Other effects of the composition include preserving vitality, health, physical vigour, quality of life, and delaying the signs of aging. Physical vigour includes the pet having energy and being active. Physical vigour can be measured by the animals overall energy level, mobility, appetite and playfulness.

According to a fourth aspect of the invention, there is provided a method of preserving mobility or preventing decline in mobility in an animal, in particular in a cat or a dog comprising administering, to said animal, the composition as defined in the first aspect of the invention. Signs of decreased mobility in a pet include lifestyle and behavioural changes such as a reduced ability or willingness to jump or run/walk, increased sleep, and difficulty going up and down stairs.

According to a fifth aspect of the invention, there is provided a method of delaying aging in an animal, in particular in a cat or a dog comprising administering, to said animal, the composition as defined in the first aspect of the invention. Signs of aging can include a general decrease in energy, slower movements, reduced hearing, reduced eyesight etc.

According to a sixth aspect of the invention a method of making a composition, as defined in the first aspect of the invention, comprising mixing together the ingredients into a composition e.g. in a tote tumbler to produce a powder, pellet or a paste or as described above. The product can in all other ways be produced by processes known in the art. The composition as defined in the first aspect of the invention may be added prior to or following heating or cooking of one or more of the other ingredients.

The invention will now be described with reference to the following, non-limiting examples:

Assistance provided by Elodie le Tilly, Laurent Beck and Jerome Guicheux from the Laboratory for OsteoArticular and Dental Tissue Engineering (LIOAD), in performing the experiments below was greatly appreciated.

### EXAMPLE 1

In vivo animal model study:

A pre-clinical trial was run to evaluate the efficacy of a composition containing chrondroitin, hydrolysed collagen and green tea extract on the progression of osteoarthritis in mice. Eighteen month old C57BL/6 male mice were given free access to food and water and a 12 h/12 h light/dark cycle for a week to acclimatise.

The prevalence of osteoarthritis is not 100% in this animal model therefore groups of 15 mice were tested in order to obtain statistical significance. Three groups were studied:
Healthy control group of 15 6 month old mice fed with control diet.
Control group of 15 18 month old mice fed with control diet.
Test group of 15 18 month old mice fed with a diet containing a blend of chrondroitin, hydrolysed collagen and green tea extract.

Mice were offered 6 g of food a day and only consumed 3 g of food per day. The dose of active compounds added to the diet per day were:
Green tea extract: 0.15% (146.5 mg/kg body weight/day)
Hydrolysed collagen: 0.5% (488.5 mg/kg body weight/day)
Chondroitin: 0.1% (97.5 mg/kg body weight/day)

The choice of mice as a model was to mimic a long period of life in dogs; 2 months in mice is equivalent to approximately 1 year in dogs. To convert dog doses into mice doses, the following equation from the FDA was used:

Dose in mice (mg/Kg)=dose in dog (mg/Kg)×(dog body weight (Kg)/mouse body weight (Kg))^{0.33}

For this study it was considered that dog body weight was 30 Kg and mice body weight was 30 g.

Evaluation:

Radiographic pictures of the hind legs (antero-posterior and lateral views) were taken using MX-20 DC-12 device (Faxitron Biotics LLC, Tucson, Ariz., USA) to evaluate structural damage in the joints of the mice. Mice were anesthetized by injecting intra-peritoneal a mixture of Ketamine (Ketamine 1000™, Virbac, France) at the dose of 75 mg/Kg and Xylazine (Rompun™, Bayer, France) at a dose of 5 mg/Kg. Pictures were taken at the beginning of the study and every five weeks until natural death. Using a blinded procedure, the severity of the osteoarthritis was scored from each image using multiple grade criteria as set out in Table 1 and 2 below.

TABLE 1

| | | |
|---|---|---|
| Circumference of medial | <1200 pixels | 0 |
| and lateral menisci | 1200-2000 | 1 |
| | >2000 pixels | 2 |
| Number of visible | None | 0 |
| osteophytes | 1 | 1 |
| | >1 | 2 |
| Structural modifications of | None | 0 |
| subchondral bone (sclerosis) | Low-Moderate | 1 |
| | Moderate-High | 2 |
| Width of the joint space | Normal | 0 |
| | Reduced | 1 |
| Calcifications of tendons | None | 0 |
| | 1 site | 1 |
| | >1 site | 2 |
| Total Arthrosis score | Total | 9 |

TABLE 2

| Radiological scoring | OA grade |
|---|---|
| 0-1 | 0 |
| 2-3 | 1 |
| 4-5 | 2 |
| 6-7 | 3 |
| 8-9 | 4 |

Mouse gait was measured using the CatWalk™ System (Noldus Information Technology, Netherlands) which records mice footprints with a light system and a high frequency video camera. Using the analysing software, CatWalk XT™ 10, fine variations of mice gait which is likely to happen in osteoarthritis was measured. Gait analysis was performed at the beginning of the study and measured every five weeks until death. Each mouse was allowed to walk freely from one side of the walkway to the other. At each contact of the paw with the glass plate, the LE light was reflected down through the glass floor and recorded by a camera. Reliable recordings were obtained by training mice daily to cross the walkway for 7 days before actual gait analysis.

Results:

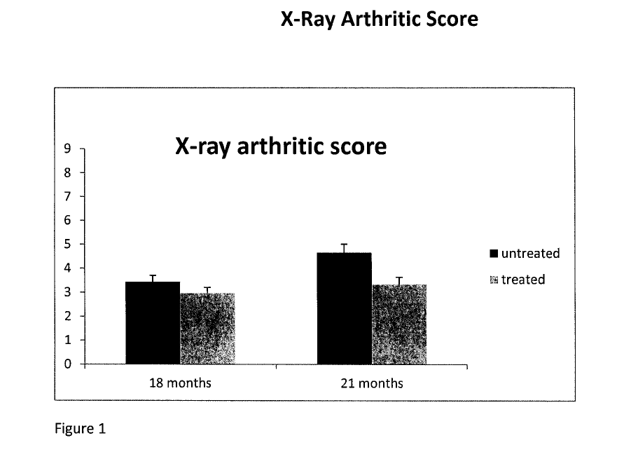

FIG. 1 shows the Radiological scoring of arthrosis at the beginning of the study (18 month old mice) and after 3 months of mice begin treated with or without the composition containing chondroitin, hydrolysed collagen and green tea extract. The composition containing chondroitin, hydrolysed collagen and green tea extract was able to prevent the evolution of arthrosis.

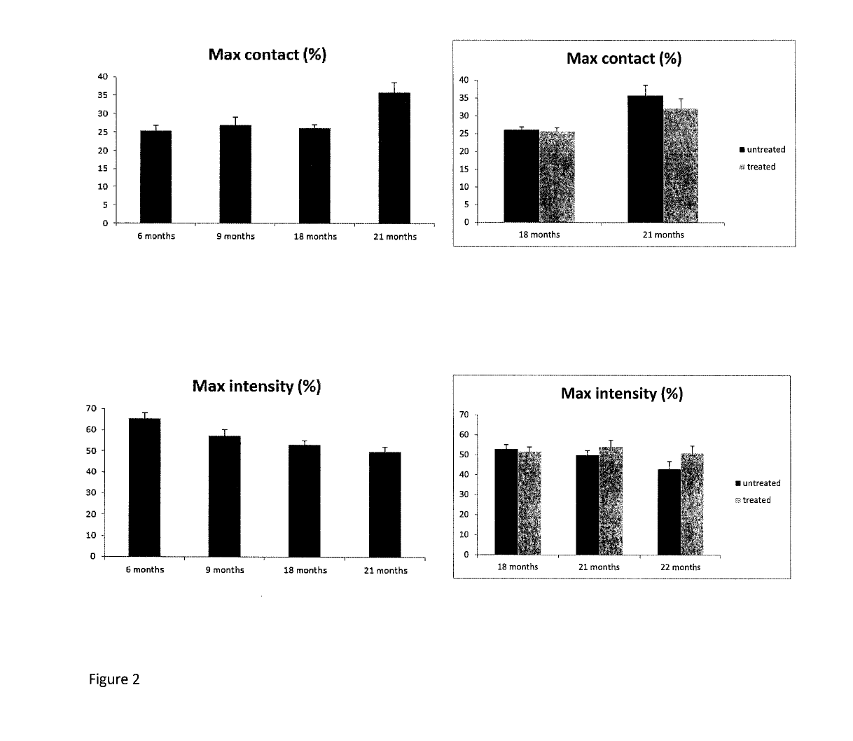

FIG. 2 shows mobility measured using the CatWalk™ System in mice at 6 months, 9 months, 18 months and 21 months old, treated with or without the composition containing chondroitin, hydrolysed collagen and green tea extract. The composition containing chondroitin, hydrolysed collagen and green tea extract was able to prevent the decrease in mobility.

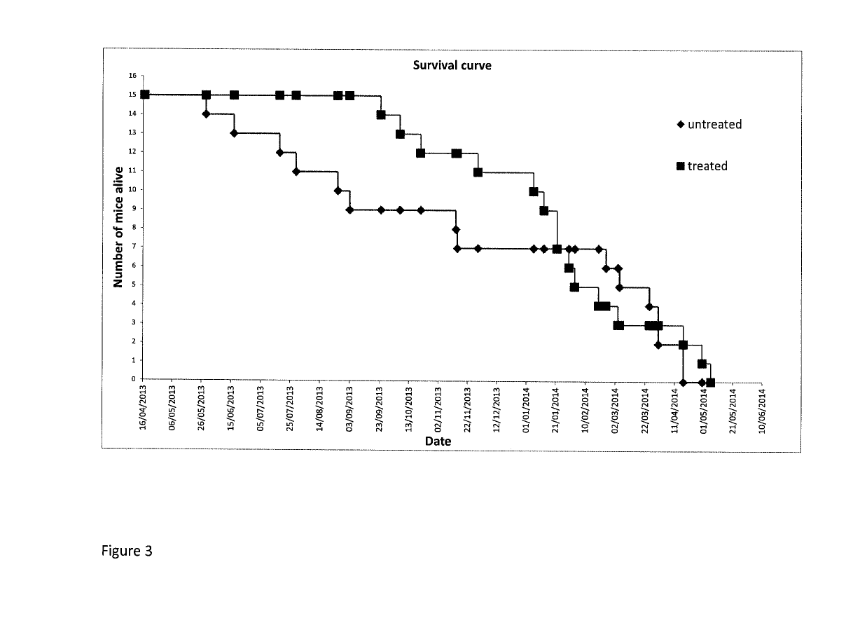

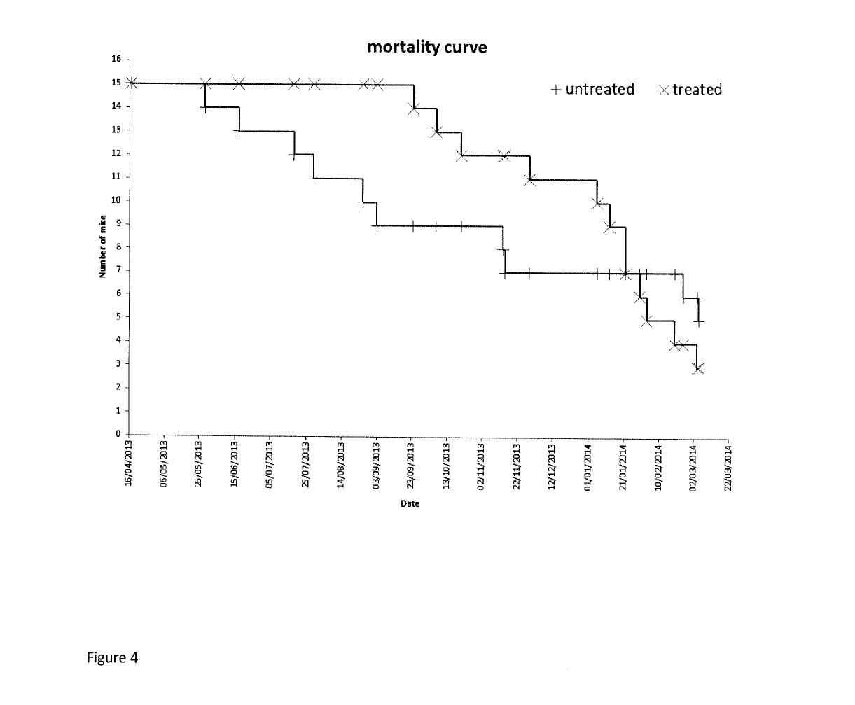

FIGS. 3 and 4 shows the date of death of mice treated with or without the composition containing chondroitin, hydrolysed collagen and green tea extract. The composition containing chondroitin, hydrolysed collagen and green tea extract increased overall survival in mice.

Generally with aging arthrosis appears in joints, which lead to pain and reduction of mobility. The composition containing chondroitin, hydrolysed collagen and green tea extract delays arthrosis appearance, prevents mobility decline and delays natural death in again mice. The mechanism by which the cocktail acts is unknown. We hypothesis that green polyphenols prevent oxidation and collagen hydrolysed and chondroitin sulphate prevent cartilage catabolism and/or increase cartilage synthesis.

This is the first time that it has been shown that a composition containing chondroitin, hydrolysed collagen and green tea extract prevents arthrosis and delays natural death. Another advantage is the cost of the composition is relatively low; indeed the three compounds are cheaper than a lot of known compounds efficient in the treatment of arthrosis such as curcumine. In addition, these compounds are thermostable unlike many other compounds known in the art that treat arthritis. An unexpected benefit of the invention is the effect of extending the lifespan of the animal.

### EXAMPLE 2

In Vitro Study:

An in vitro study was run to evaluate the efficacy of a composition containing chondroitin, hydrolysed collagen and green tea extract on Rabbit Articular Chondrocytes (RAC). RAC were cultured in DMEM supplemented with 10% Fetal Calf Serum, glutamine and 1% penicillin/streptomycin (Gibco, Life Technologies AG, Switzerland) at 37° C. in an incubator with an atmosphere of 5% CO₂.

RAC were plated in a 96 well plate (10⁵ cells/cm²). The medium was replaced with fresh medium containing each compound chondroitin, hydrolysed collagen and green tea extract or a mixture of all three compounds for 24 hours prior to stimulation with IL-1β (10 ng/ml) (Merck Millipore, USA). IL-1β is a proinflammatory cytokine linked to the pathogenesis of arthritis. RAC were then incubated for 24 hours, after which Nitric Oxide (NO) and Prostaglandin E2 (PGE2) production was measured as markers of arthritis.

RAC were plated in 6 well plates (3×10⁵ cells/cm2). The medium was replaced with fresh medium containing each compound chondroitin, hydrolysed collagen and green tea extract or a mixture of all three compounds for 24 hours prior to stimulation with IL-1β (10 ng/ml) (Merck Millipore, USA). RAC were then incubated for for 48, hours after which RNA expression of cyclooxgenase-2 (COX-2), inducible isoform Nitric oxide Synthase (iNoS) and Matrix Metallopeptidase-13 (MMP13) was measured as markers of arthritis.

Evaluation:

Cellular viability was measured using an MTS assay (Cell Titer 96™ MTS, Promega, France) as per manufacturer's instructions. Cells were also counted using a Malassez cell and cellular viability was assessed using trypan blue exclusion dye technique, which colours dead cells in blue.

NO and PGE2 production was measured using Cayman Chemicals (Bertin Pharma, France) following the manufacturer's protocol. NO production was estimated spectrophotometrically by measuring the accumulation of nitrites in the culture supernatants by Griess reaction using a standard curve prepared with sodium nitrite. PGE2 was estimated using a highly sensitive and specific Enzyme Immunoassay Kit. RNA extraction was performed by extracting RNA using the NucleoSpin™ RNA II Kit (Macherey-Nagel, Hoerdt, France) as per manufacturer's instructions. RNA was reverse-transcribed with SupersScript III (Life Technologies) and real-time polymerase chain reaction (RT-PCR) was performed with SYBR Select mix (Life Technologies). Primer sequences COX-2, iNoS and MMP13 was used to quantify the relative RNA expression. Primer sequence for GAPDH was used as the reference gene. Table 3 lists the primer sequences used in Example 2.

TABLE 3

| | Reference | | | Size |
|---|---|---|---|---|
| Amplified gene | Gene Bank | Primer sequences | | (bp) |
| COX2 | NM_001082388.1 | Forward: | GGAAGCGCTCTACGGCGACA | 3314 |
| | | Reverse: | CCCCAAAGATGGCATCCGGGC | |
| iNos | AF198443.1 | Forward: | TGACGTCCAGCGCTACAATA | 247 |
| | | Reverse: | TGCGTCTCCAGTCCCATC | |
| MMP13 | NM_001082037.1 | Forward: | TTTTGAAGACACGGGCAAG | 3124 |
| | | Reverse: | TCATCATAGCTCCAGACTTGGTT | |
| GAPDH | NM_001082253 | Forward: | AGAACGGGAAGCTGGTCAT | 1282 |
| | | Reverse: | TTGATGTTGGCGGGATCT | |

The experimental results from Example 2 are presented in Table 4 below.

TABLE 4

| | | PGE2 production | NO Production | COX2 | iNoS | MMP13 |
|---|---|---|---|---|---|---|
| Treatment | Treatment | (ng/ml) | (μM) | Expression | Expression | Expression |
| Untreated | Untreated | 8.4 | 14.0 | 1.0 | 1.0 | 1.0 [symbol] |
| Untreated | IL-1β (10 ng/ml) | 91.1 | 24.9 | 9.9 | 6.2 | 378.9 [symbol] |
| Green tea extract (50 μM) | IL-1β (10 ng/ml) | 10.3 | 19.4 | 4.3 | 6.7 | 79.3 [symbol] |
| Collagen (0.5 mg/ml) | IL-1β (10 ng/ml) | | | 7.1 | 3.9 | 240.1 [symbol] |
| Chondroitin (200 μg/ml) | IL-1β (10 ng/ml) | | | 10.7 | 5.3 | 451.0 [symbol] |
| Green tea extract (50 μM) | IL-1β (10 ng/ml) | 6.2 | 21.8 | 5.5 | 6.2 | 66.3 [symbol] |
| and Collagen (0.5 mg/ml) | | | | | | |
| and Chondroitin | | | | | | |
| (200 μg/ml) | | | | | | |

[symbol]

indicates data missing or illegible when filed

Rabbit Articular Chondrocytes (RAC) were incubated with the different compounds separately or blended together to evaluate their cytotoxicity. Both MTS measurements and Trypan blue exclusion tests did not reveal any cellular toxicity after 24 and 48 hours of incubation with green tea extract, chondroitin or hydrolysed collagen throughout a large range of concentration (data not shown).

A pre-treatment with green tea extract, as well as with the mix of green tea extract, chondroitin and hydrolysed collagen caused a significant and dose-dependent inhibition of NO and PGE2 production (Table 4) (P<0.05). Hydrolysed collagen decreases the expression of the different genes, whereas chondroitin alone seems to have no effect. On the other hand, green tea extract, as well as the mix of green tea extract, chondroitin and hydrolysed collagen inhibited the expression of COX2 and MMP13, but had no effect on iNos expression (Table 4).

The mix of green tea extract, chondroitin and hydrolysed collagen reversed the effect of IL1-□ on inflammatory cytokines and catabolic enzymes, such as NO, PGE2 and MMP13, thereby demonstrating their anti-inflammatory and anti-catabolic properties on an in vitro model of the disease.

1. A composition comprising green tea extract, collagen and chondroitin for use in preventing or treating arthritis in an animal, in particular in a cat or a dog.

2. The composition for use as claimed in claim 1 wherein arthritis is osteoarthritis.

3. A composition comprising green tea extract, collagen and chondroitin for use in increasing life expectancy in an animal, in particular in a cat or a dog.

4. A composition comprising green tea extract, collagen and chondroitin for use in delaying aging in an animal, in particular in a cat or a dog.

5. The composition for use according to claims 1 to 4 wherein the composition comprises any one or more of the ingredients present in the following ranges: green tea extract at 15 mg to 5 g, hydrolysed collagen at 50 mg to 17 g, and chondroitin at 10 mg to 4 g.

6. The composition for use according to claims 1 to 5 wherein the composition is in the form of a food.

7. A method of treating arthritis in a cat or a dog comprising administering, to an animal, in particular in a cat or a dog, a composition comprising green tea extract, collagen and chondroitin.

8. The method for treating arthritis as claimed in claim 7 wherein arthritis is osteoarthritis.

9. A method of increasing life expectancy in an animal, in particular in a cat or a dog comprising administering to said animal, to a cat or a dog, a composition comprising green tea extract, collagen and chondroitin.

10. A method of delaying aging in an animal, in particular in a cat or a dog comprising administering, to said animal, a composition comprising green tea extract, collagen and chondroitin.
